(19) United States
(12) Patent Application Publication
(10) Pub. No.: US 2023/0026386 A1
(43) Pub. Date: Jan. 26, 2023

(54) Sanitary Undergarment

(71) Applicant: Jazz Burney, Citrus Heighs, CA (US)

(72) Inventor: Jazz Burney, Citrus Heighs, CA (US)

(21) Appl. No.: 17/532,003

(22) Filed: Nov. 22, 2021

Related U.S. Application Data
(60) Provisional application No. 63/224,644, filed on Jul. 22, 2021.

(51) Int. Cl. A61F 13/49; A61F 13/47; A61F 13/511; A61F 13/56

(52) U.S. Cl. CPC A61F 13/49058; A61F 13/49003; A61F 13/47; A61F 13/51113; A61F 13/5633; A61F 13/5611; A61F 2013/424

(57) ABSTRACT

The present invention relates to a sanitary diaper or undergarment having a body shield that may be made of a plurality of materials and may be infused with a plurality of substances that have anti-fungal, anti-bacterial and moisturizing properties. The body shield may be hypoallergenic, scented or non-scented. The interior of the body shield has an adhesive flap that can be applied to the genital area or hip area of the wearer, in order to seal the genital area or hip areas of the wearer from any human waste that accumulates within the diaper. As a result, the device greatly reduces and prevents the occurrence of diaper rash and other skin irritations that can occur from wearing a diaper for prolonged periods of time.

### CROSS-REFERENCE TO RELATED APPLICATION

The present application claims priority to, and the benefit of, U.S. Provisional Application No. 63/224,644, which was filed on Jul. 22, 2021, and is incorporated herein by reference in its entirety.

### FIELD OF THE INVENTION

The present invention relates generally to the field of sanitary undergarments. More specifically, the present invention relates to a sanitary diaper device having a body with a first tab, a second tab, an interior surface, a front surface, a first adhesive tab, a second adhesive tab and at least one elastic band. The body of the undergarment may be made of a plurality of materials, such as woven, spun-woven, bonded or the like, and may be infused with a plurality of substances that have anti-fungal, anti-bacterial, moisturizing and other properties. In addition, the undergarment may be hypoallergenic and scented or non-scented. The interior of the undergarment further has an adhesive flap that can be applied to the genital area or hip area of the wearer, in order to seal the genital area of the wearer from the rest of the diaper and any urine or feces that accumulate in the diaper. As a result, the undergarment greatly reduces and prevents the occurrence of rashes or other skin irritations that can occur from wearing an undergarment for prolonged periods with urine and/or feces within the undergarment. Accordingly, the present disclosure makes specific reference thereto. Nonetheless, it is to be appreciated that aspects of the present invention are also equally applicable to other like applications, devices and methods of manufacture.

### BACKGROUND

Infants and others that wear diapers or sanitary undergarments may regularly experience a rash or skin ailments, which is an irritation of the skin covered by the sanitary undergarment. Such skin rashes can be caused by the enzymes in feces that can irritate the skin of the infant or individual as the feces sit within the diaper. Urine may also cause a rash or irritation, as ammonia is released as urine breaks down, which causes the pH of the skin to rise and further release the enzymes of any feces within the diaper. Such rashes can also be caused by chafing from friction or rubbing of the diaper or undergarment. As a result, such rashes are extremely unpleasant for the infant or other individual wearing the undergarment. In addition, feces and urine that sit within a diaper or sanitary undergarment for a long period of time may begin to smell, which is undesirable for other nearby individuals.

Therefore, there exists a long-felt need in the art for an improved diaper or sanitary undergarment. There also exists a long-felt need in the art for a sanitary diaper or undergarment that prevents rashes and other forms of skin irritation. Further, there exists a long-felt need in the art for a sanitary diaper or undergarment that prevents feces and urine within the diaper from contacting the genital area and other skin areas of the wearer. In addition, there exists a long-felt need in the art for a sanitary diaper or undergarment that is hypoallergenic and contains compounds that reduce skin irritation.

The subject matter disclosed and claimed herein, in one embodiment thereof, comprises a sanitary diaper or undergarment. The sanitary undergarment includes a body that has a first tab, a second tab, an interior surface, a front surface, a first adhesive tab, a second adhesive tab and at least one elastic band. In differing embodiments, the body of the device may have a plurality of materials which may be infused with different substances that have anti-fungal, anti-bacterial, moisturizing and other properties. The sanitary undergarment may be hypoallergenic, and scented or non-scented. The interior of the undergarment further has an adhesive flap that can be applied to the genital area, hip area or other exposed skin of the wearer, in order to seal the exposed skin area of the wearer from the rest of the diaper or sanitary undergarment, and any urine or feces within the diaper or undergarment. As a result, the undergarment greatly reduces and prevents the occurrence of rashes and other irritations that can occur from wearing a diaper or sanitary undergarment for prolonged periods of time when the diaper or undergarment contains any accumulation of urine or feces within the diaper or undergarment.

In this manner, the sanitary diaper or undergarment of the present invention accomplishes all of the forgoing objectives and provides an improved diaper. Further, the sanitary diaper or undergarment of the present invention is particularly advantageous as it prevents rashes and other forms of skin irritation by sealing the exposed skin areas of the wearer from the accumulation of waste materials. To accomplish this, the sanitary undergarment prevents feces and urine within the diaper or undergarment from contacting the exposed skin and genital area of the wearer via a unique flap configuration. In addition, the device is hypoallergenic and contains compounds that reduce skin irritation to further prevent diaper rash and other skin irritations.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a sanitary diaper or undergarment. The sanitary undergarment includes a body that has a first tab, a second tab, an interior surface, a front surface, a first adhesive tab, a second adhesive tab and at least one elastic band. In differing embodiments, the body of the sanitary undergarment may be made of a plurality of materials and may be infused with a plurality of substances that have anti-fungal, anti-bacterial, moisturizing and other properties. Further, the material of the body may be hypoallergenic and may be scented or non-scented.

The body of the sanitary undergarment is generally U-shaped and has a first tab and second tab on the first end of the body. The tabs of the body each receive an adhesive tab that is located on the second end of the body. Each adhesive tab also has an adhesive bottom surface that allows each tab to attach to the first tab and second tab, to secure the sanitary undergarment around the waist and legs of an infant or other individual. The body may further include at least one elastic band to aid in securing the sanitary undergarment to the waist and legs of a wearer. The at least one band may be integrated within the material of the body, and may be positioned around the continuous waist opening and/or the continuous leg openings of the body. In this manner, the sanitary undergarment is better secured to the waist and legs of the wearer.

The interior of the body may also have a soft padding. The padding ensures comfort for the wearer and may be absorbent in order to absorb urine and/or feces. The portion of the interior nearest the first end of the body has a protective flap. The flap may be absorbent or waterproof, water-resistant and breathable. The inner surface of the body of the flap has a first adhesive tab and a second adhesive tab, and the adhesive of both tabs is a skin-safe adhesive. However, the entire perimeter of the inner surface of the sanitary garment may be additionally or alternatively be adhesive. Therefore, the flap can be adhered to the predetermined skin areas, such as the genital area and or hip area of an infant or other wearer. As a result, the exposed skin or genital area of the wearer is effectively sealed from the rest of the interior, and any feces or urine that fills the interior cannot come into contact with the genital or skin area of the wearer, therefore preventing rash, skin irritations and infections caused by exposure to urine and/or feces.

Accordingly, the improved sanitary diaper or undergarment of the present invention is particularly advantageous, as it prevents rashes and other forms of skin irritation by sealing the exposed skin and genital area of the wearer from the rest of the body of the sanitary garment. In doing so, the device prevents feces and urine within the diaper or sanitary garment from contacting the genital or skin area of the wearer. In addition, the device is hypoallergenic and contains compounds that reduce skin irritation to further prevent rashes and other irritations.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of one potential embodiment of a sanitary diaper or undergarment of the present invention in accordance with the disclosed architecture;

FIG. 2 illustrates a front perspective view of one potential embodiment of a sanitary diaper or undergarment of the present invention in a semi-open position in accordance with the disclosed architecture; and

FIG. 3 illustrates a perspective view of the interior of one potential embodiment of a sanitary diaper or undergarment of the present invention in an open position in accordance with the disclosed architecture.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for an improved diaper or sanitary undergarment. There also exists a long-felt need in the art for a sanitary diaper or undergarment device that prevents rashes and other forms of skin irritation. Further, there exists a long-felt need in the art for a sanitary diaper or undergarment that prevents feces and urine within the diaper from contacting the genital area of the wearer. In addition, there exists a long-felt need in the art for a sanitary diaper or undergarment that is hypoallergenic and contains compounds that reduce skin irritation.

The present invention, in one exemplary embodiment, is comprised of a sanitary diaper or undergarment. The device is primarily comprised of a body that has a first tab, a second tab, an interior surface, a front surface, a first adhesive tab, a second adhesive tab and at least one elastic band. In differing embodiments, the body of the undergarment may be made of a plurality of materials and may be infused with various substances. The materials or substances may have anti-fungal, anti-bacterial, and moisturizing properties and may be hypoallergenic and may be scented or non-scented.

The body is generally U-shaped to fit around the lower portion of a human torso and has a first tab and second tab located on the first end of the body. The tabs of the body each have an adhesive tab that is located on the second end of the body. Each adhesive tab also has an adhesive bottom surface, that allows each tab to be attached to the first tab and second tab to secure the device around the waist and legs of an infant or other wearer. The body may further include at least one elastic band to aid in securing the device to the waist and legs of the individual wearer. The at least one band is preferably integrated within the material of the body, and may be positioned around the continuous waist opening and/or the continuous leg openings of the body. Accordingly, the device is better secured to the waist and legs of the wearer, who may be an infant, child, adolescent or an adult.

The interior of the body may have a soft padding that ensures comfort for the wearer, and may be absorbent in order to absorb urine, feces or other fluids. The portion of the interior nearest the first end of the body has a protective flap. The flap may be absorbent or waterproof, water-resistant and breathable. The inner surface of the body of the flap has a first adhesive tab and a second adhesive tab, and the adhesive of both tabs is a skin-safe adhesive. However, the entire perimeter of the inner surface may be additionally or alternatively be adhesive. Therefore, the flap can be adhered to the arear surrounding the genital area and/or hip area of a wearer. As a result, the genital area of the wearer is effectively sealed from the rest of the interior and any feces, urine or other fluids that fill the interior and come into contact with the skin around the genital area of the wearer, therefore preventing rash, irritations and infections caused by urine, feces and other fluids.

Accordingly, the improved sanitary diaper device of the present invention is particularly advantageous as it prevents rashes, infections and other forms of skin irritation by sealing the genital area and surrounding skin of the wearer from the rest of the interior of the sanitary garment. In doing so, the undergarment structure prevents feces, other waste material and urine within the diaper or undergarment from contacting the genital area and surrounding skin of the infant, child or adult wearer via the flap. In addition, the undergarment or diaper is hypoallergenic, and contains compounds that reduce skin irritation to further prevent rashes and irritations.

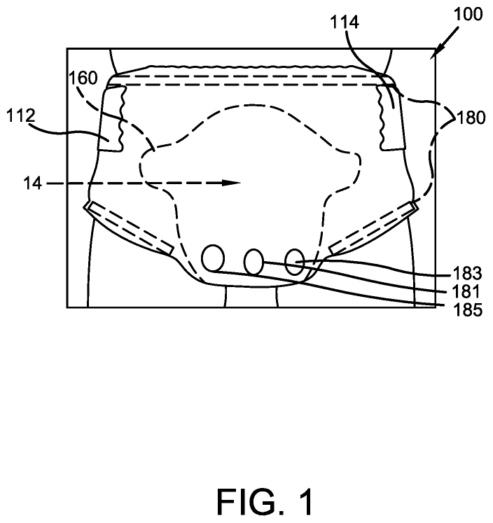

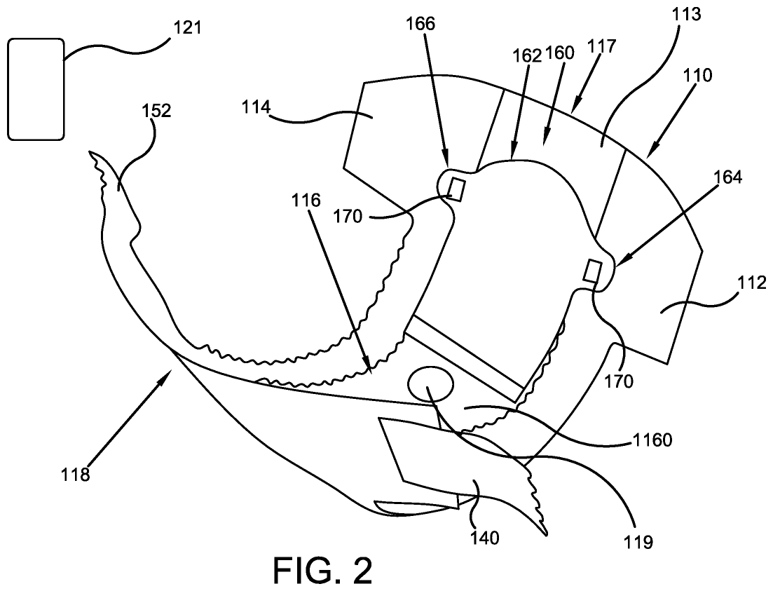

Referring initially to the drawings, FIG. 1 illustrates a front perspective view of one potential embodiment of a sanitary diaper or undergarment 100 of the present invention in accordance with the disclosed specification. The undergarment or diaper 100 is primarily comprised of a body 110 that has a first tab 112, a second tab 114, an interior surface 116, a front surface 120, a first adhesive tab 140, a second adhesive tab 150 (see FIG. 2) and at least one elastic band 180. In differing embodiments, the body 110 of the undergarment 100 may be made of a plurality of materials such as, but not limited to, wood pulp, sodium polyacrylate fibers, plastic (ex. cellulose, polypropylene, polyethylene etc.), tissue paper, non-woven fabric, non-permeable film or any other traditional diaper material known in the art or combination thereof. In addition, the body 110 and/or all components of the diaper or undergarment 100 may be infused with a plurality of substances unless otherwise stated, including but not limited to: aloe vera, zinc oxide, calendula, lanolin, petroleum, and other moisturizing, anti-fungal, anti-bacterial compounds or combinations thereof. Further, the material of the body 110 may be hypoallergenic and may be scented or non-scented. The body 110 may also include one or more essential oils such as peppermint, lavender, tea tree, chamomile and combinations thereof, to help soothe any skin irritation.

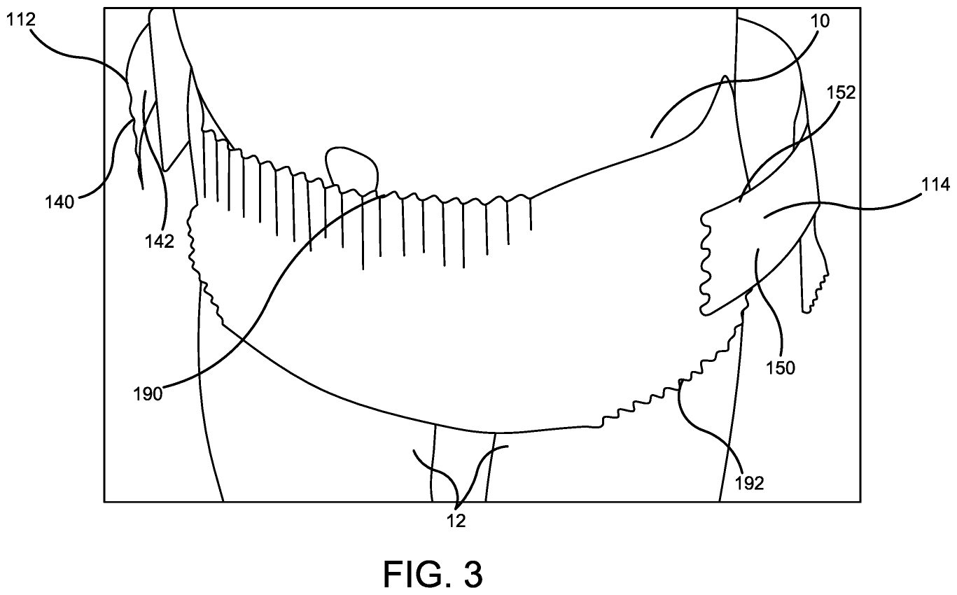

The exterior of the diaper or sanitary garment may also contain change indicators 181, 183 and 185 which can indicate the condition of the diaper or sanitary garment. The change indicators can be triggered by the sensor 119 (see FIG. 3) or may be triggered by moisture levels, weight, bacteria or chemical levels or combinations thereof.

FIG. 2 illustrates a front perspective view of one potential embodiment of a sanitary diaper or undergarment of the present invention in a semi-open position in accordance with the disclosed specification. The body 110 is generally U-shaped and configured to fit to the lower portion of the human torso and between the legs of the individual. The body 110 has a first tab 112 and second tab 114 on the first end 117 of the body 110. Each tab 112, 114 has an adhesive tab area 140, 150 extending from the second end or back panel or portion 118 (see FIG. 3) of the body 110 that has an adhesive bottom surface 142, 152, that allows each tab 140, 150 to attach to the first tab 112 and second tab 114 (or to the front surface 120) to secure the device 100 around the waist 10 and legs 12 of a wearer. To further secure the undergarment 100 to the waist 10 and legs 12 of a wearer, the body 110 may have at least one elastic band 180. The at least one band 180 may be integrated within the material of the body 110 and may be positioned around the waist opening 190 and/or the leg openings 192, such that the device 100 is better secured to the waist 10 and legs 12 of the wearer.

FIG. 3 illustrates a perspective view of the interior 116 of one potential embodiment of a sanitary diaper or undergarment 100 of the present invention in an open position in accordance with the disclosed specification. The interior 116 of the body 110 preferably includes a soft padding 1160 that ensures comfort for the wearer. The interior region 116 may also include a sensor 119 for detecting wetness, or other chemicals that may be triggered by an infection. The sensor 119 may be an RF sensor, and the presence of wetness or another trigger causes a different signal to be emitted from the sensor 119, which may be picked up by a smart device 121 having a previously downloaded software application to interact with the sensor 119. The sensor 119 is positioned in a portion of the undergarment 100 in order to detect one or more conditions including, but not limited, to wetness, temperature, chemicals, bacteria or combinations thereof. In one embodiment, the padding 1160 may be absorbent. The portion of the interior 116 nearest the first end 117 further includes a protective flap 160. The body of the diaper or garment has an expansible area 113 to accommodate different sizes of individuals. In differing embodiments, the flap 160, which is positioned in the interior of the diaper or undergarment may be absorbent or waterproof, water-resistant and breathable. The inner surface 168 of the shield 162 of the flap 160 includes a first adhesive tab 164 and a second adhesive tab 166, and the adhesive of both tabs is a skin-safe adhesive. Additional adhesive areas 170 may be provided to hold the shield 162 in position against the body of the wearer. However, it is contemplated that in addition to or in lieu of both adhesive tabs 164, 166, the entire perimeter of the inner surface of the shield 168 may be adhesive to create a seal around the interior to prevent any leakage. The shield 168 may be removable, repositionable and/or reusable, so that the shield 168 can be cleaned and treated with additional substances or materials for use in another diaper or sanitary garment. In addition, by providing a shield that can be moved or changed, the shield 168 can be adapted to fit the particular individual, and is not permanently adhered to the diaper or sanitary garment. In this manner, the flap 160 can be adhered to the genital area and surrounding area 14 and/or hip area 16 of an infant or other wearer (shown in FIG. 1). To this effect, the genital area and surrounding area 14 of the wearer is effectively sealed from the rest of the interior 116. In this manner, any feces, other fluids or urine that fills the interior 116 is prevented from coming into contact with the genital area and surrounding area 14 of the wearer and, as a result, rashes, skin irritations and infections are prevented.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “sanitary diaper” and “diaper or undergarment” are interchangeable and refer to the sanitary diaper device 100 of the present invention.

Notwithstanding the forgoing, the sanitary diaper or undergarment 100 of the present invention and its various components can be of any suitable size and configuration as is known in the art without affecting the overall concept of the invention, provided that they accomplish the above-stated objectives. One of ordinary skill in the art will appreciate that the size, configuration and material of the sanitary diaper or undergarment 100 as shown in the FIGS. are for illustrative purposes only, and that many other sizes and shapes of the sanitary diaper or undergarment 100 are well within the scope of the present disclosure. Although the dimensions of the sanitary diaper or undergarment 100 are important design parameters for user convenience, the sanitary diaper or undergarment 100 may be of any size, shape and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A sanitary undergarment comprising:
a body comprised of a generally U-shape and first and second top ends which are connected to one another by adhesive tabs disposed on an end edge of one of the first and second ends, and wherein the body has a front surface and an interior surface;
a cut out area defining leg openings disposed centrally of the body, each of the cut out areas having an elastic band for securing the cut out area around legs of a wearer; and
a shield disposed over an area covering a genital region of the wearer.

2. The sanitary undergarment as recited in claim 1, wherein the shield is one of removable, reusable and repositionable and is positioned on the interior surface of the body.

3. The sanitary undergarment as recited in claim 2, wherein the shield can be moved to accommodate different wearers.

4. The sanitary undergarment as recited in claim 1, wherein the shield is adhesively attached to the genital region of the wearer.

5. The sanitary undergarment as recited in claim 4, wherein the shield prevents the genital region of the wearer from coming into contact with human waste or other fluids.

6. The sanitary undergarment as recited in claim 1, wherein a sensor is provided on the interior surface of the body in an area to detect conditions of the body.

7. The sanitary undergarment as recited in claim 6, wherein the conditions include wetness, temperature, chemicals, bacterial or combinations thereof.

8. The sanitary undergarment as recited in claim 6, wherein the sensor is an RF sensor.

9. The sanitary undergarment as recited in claim 8, wherein the sensor is connected to a smart phone having a software application for detecting signals from the sensor.

10. The sanitary undergarment as recited in claim 1, wherein change indicators are provided on the front surface of the body.

11. The sanitary undergarment as recited in claim 10, wherein the change indicators are triggered by at least one of moisture levels, weight, bacterial, chemical levels or combinations thereof.

12. A diaper comprising:
a diaper body being sized and configured to fit around a lower torso portion of a human body;
the diaper body held in position by first and second adhesive tabs extending from a rear surface of the diaper body to a front surface of the diaper body;
a pair of leg openings centrally disposed in the diaper body, each of the leg openings having an elastic band to hold the diaper body to a wearer; and
a shield insertable into the diaper body and positioned on an interior surface of the diaper body, the shield connected to the diaper body by adhesive, and the shield is positioned within the diaper body so as to cover a genital region of a wearer of the diaper.

13. The diaper as recited in claim 12, wherein the shield is treated with at least one of aloe vera, zinc oxide, calendula, lanolin, petroleum, moisturizing, anti-fungal, and anti-bacterial compounds.

14. The diaper as recited in claim 12, wherein the shield is treated with one or more essential oils such as peppermint, lavender, tea tree, chamomile and combinations thereof.

15. The diaper as recited in claim 12, wherein the shield is provided with an adhesive extending around a perimeter to hold the shield in position over the genital region of the wearer.

16. The diaper as recited in claim 12, wherein the diaper is provided with one of a sensor and change indicators.

17. The diaper as recited in claim 16, wherein the sensor is a RF sensor and is connected to a smart phone having a software application to receive signals from the RF sensor.

18. The diaper as recited in claim 17, wherein the sensor detects conditions relating to wetness, temperature, chemicals, bacterial or combinations thereof.

19. A sanitary diaper configuration comprising:
a diaper having a generally U-shaped body with a first end and a second end and a pair of leg cut outs with each of the leg cut outs having an elastic band, the body having an interior surface and an exterior surface and a pair of adhesive closure tabs disposed on one of the first end and second end for closing the body around a torso of a wearer;
a shield body positioned on the interior surface and in an area over a genital region of the wearer, the shield is adhesively secured to the wearer and is removable and repositionable; and
the shield is hypoallergenic and is treated with at least one of aloe vera, zinc oxide, calendula, lanolin, petroleum, moisturizing, anti-fungal, and anti-bacterial compounds.

20. The sanitary diaper configuration as recited in claim 19, wherein the shield prevents the genital region of the wearer from coming into contact with human waste or other fluids.
